(19) United States
(12) Patent Application Publication
(10) Pub. No.: US 2011/0098597 A1
(43) Pub. Date: Apr. 28, 2011

(54) MICROFLUIDIC SAMPLERS AND METHODS FOR MAKING AND USING THEM

(75) Inventors: Christine Hsiao-Ming Wu, Santa Clarita, CA (US); Hsian-Rong Tseng, Los Angeles, CA (US); Guodong Sui, Boca Raton, FL (US); Cheng-Chung Lee, Irvine, CA (US); Hong Dun Lin, Los Angeles, CA (US)

(73) Assignee: The Regents of the University of California, Oakland, CA (US)

(21) Appl. No.: 12/090,069

(22) PCT Filed: Oct. 13, 2006
(86) PCT No.: PCT/US06/40276

§ 371 Date: Aug. 15, 2008

Related U.S. Application Data
(60) Provisional application No. 60/726,110, filed on Oct. 13, 2005.

(51) Int. Cl. A61B 5/157; C12M 1/26; B01L 99/00

(52) U.S. Cl. 600/573; 435/309.1; 422/504

(57) ABSTRACT

This invention provides microfluidic samplers for withdrawing one or more precise micro- or nano-liter volumes of a sample. The invention provides microfabricated automatic systems comprising integrated poly(dimethyl-siloxane) (PDMS) micro fluidics. The sample can be biological samples, including samples from animals or plants. The samples can be fluid or gas. The samples can comprise a biological fluid, such as blood, tears, cerebral spinal fluid (CSF) and the like, from a test subject such as a human or a mouse. The invention also provides methods for making and using the microfluidic samplers of the invention.

### TECHNICAL FIELD

This invention provides microfluidic samplers for withdrawing one or more precise micro- or nano-liter volumes of a sample. The invention provides microfabricated automatic systems comprising integrated poly(dimethyl-siloxane) (PDMS) microfluidics. The sample can be biological samples, including samples from animals or plants. The samples can be fluid or gas. The samples can comprise a biological fluid, such as blood, tears, cerebral spinal fluid (CSF) and the like, from a test subject such as a human or a mouse. The invention also provides methods for making and using the microfluidic samplers of the invention.

### BACKGROUND

MicroPET imaging is becoming increasingly popular in monitoring tissue biological functions in mice. One of its greatest capabilities, to quantify biological/physiological processes in vivo, remains challenging due to small blood volume (˜2 ml for a 20 g mouse) and difficulty in blood sampling. In quantitative microPET studies, a valid time-activity curve (TAC) that is normally constructed from 20 to 25 arterial blood samples in 60 minutes. This can be called an input function, and can be required for the reliable measurement of biological tissue function in terms of absolute biological units. There is a growing interest in the testing of transgenic mice as popular animal models for studying human diseases. A device capable of frequent and precise micro- and nano-liter volume blood sampling would allow determinations of input functions from mice and bring microPET imaging to new levels of precision and utility.

MicroPET imaging in small animals has recently become an important in vivo imaging technique for studying biology and for drug evaluation and development for many medical disorders, including cancer and AIDS. The number of microPET scanners in operation is growing rapidly around the world. However, the full potential of microPET technology cannot be realized without the ability to provide high precision to measurements of biological function. Blood sampling from a mouse is extremely difficult due to small blood vessel diameters (about 1 mm), small blood volume (about 2 ml in a 20 g mouse), and fast metabolism (FIGS. 1 and 2). FIG. 1, derived using manual blood sampling, illustrates that a fast transit from vena cava (RV to aorta LV took place within 3 seconds) of a PET radiotracer (in color) through a mouse heart makes input function derivation from manual blood sampling difficult. FIG. 2 shows a typical example of the first 7 seconds of the blood time activity curves that can be used for a mouse input function in a quantitative microPET study. In order to determine the shape of each curve, multiple blood sample need to be taken within a second.

Quantitative PET studies have provided enormous amounts of information about human diseases. Due to the invasiveness of arterial blood sampling, alternative methods have been explored, including the derivation of input functions from a blood pool of a left ventricular chamber of PET images. However, studies showed that such methods that were successful in deriving input functions from human PET images were not applicable to the microPET imaging of mice due to the limited microPET resolution and the small mouse heart.

To date, none of the institutes that are capable of performing microPET imaging has reported successful experience in acquiring reliably input function from animals, e.g., mice. There is a pent up demand for a high-speed, microvolumetric blood sampler around the world.

### SUMMARY

The invention provides microfabricated automatic systems—e.g., multiplexed systems,—comprising microfluidic sample devices, which in one aspect comprise integrated poly(dimethyl-siloxane) (PDMS) (or equivalent) microfluidics, and methods of making and using them. In one aspect, these products of manufacture are used to sample small quantities of a sample, e.g., a biological or an environmental sample, including liquids or gases, and each aspect of operation of the device (input, analysis, output, data analysis) can be integrated with appropriate computers and software, and in some embodiments are fully automated. In one aspect, a large number of assays (e.g., biochemical assays) can be made in parallel (simultaneously).

The invention provides microfluidic sample devices comprising: (a) at least one inlet port (e.g., a plurality of inlet ports) for a sample, including fluid or a gas samples; (b) at least one, or a plurality of switches, operably linked to the inlet (or plurality of inlet ports) by channels providing for fluidic flow to move the sample (e.g., fluid or gas sample), wherein the switches can direct a volumetrically measured (e.g., metered—which can be automated or remotely controlled) sample of fluid to a sample wells; (c) at least one, or a plurality of sample wells operably linked to the plurality of switches by channels providing for fluidic flow to move the fluid or gas sample; (d) at least one, or a plurality of volumetric metering loops operably linked to at least one of the switches by channels providing for fluidic flow to move the fluid or a gas sample, wherein the volumetric metering loop can purge sample fluid from the system; and, (e) at least one, or a plurality of evacuation (output) ports operably linked to the sample wells by channels providing for fluidic flow to move the fluid or gas sample. One, several or all of these components can be remotely controlled via electronic sensors and output to and input from a computer with appropriate software—and these operations can be completely or partially automated.

In alternative embodiments of the devices (products of manufacture) of the invention, the microfluidic sample device comprises one or more resin materials, e.g., poly(dimethyl-siloxane) (PDMS) or equivalent(s), e.g., any poly(alkyl-siloxane), or any siloxane. The microfluidic sample device switches can channel a volumetrically metered sample of fluid to sample wells (as with all operations, these operations can be completely or partially automated).

In alternative embodiments of the devices (products of manufacture) of the invention, the device is operably linked to an imaging device such that sample in the sample wells can be imaged, e.g., the imaging device can comprise a Positron Emission Tomography (PET) imaging device, pr equivalent, a camera, or any imaging or detection device (e.g., detecting radiation from radioisotopes).

In one aspect, the device is operably linked to a computer comprising software to control the amount and direction of liquid or gas sample(s) flowing into and through the device and/or to control movement of liquid or gas samples into, though and/or out of the device, including managing the flow of wash materials. The operation of the device can be partially or fully automated, and data collation and output to user can be fully automated.

In alternative embodiments of the devices (products of manufacture) of the invention, the device comprises a configuration as set forth in FIGS. 3 to 9, and FIGS. 12 to 14, or any combination thereof.

The samples processed by the microfluidic sample device of the invention can be from any source, e.g., the samples can comprise a biological fluid or gas, or an artificial fluid or gas, e.g., detecting toxins, pesticides and poisons, including synthetic substances such as nerve gases, e.g. agent VX, mustard gases (“H agents), Sarin gas and other G agents, and the like, and toxic biological agents. In one aspect, detection of agents using the devices of the invention can be used, e.g., for V agents, G agents, H agents and/or biological agents or pesticides, in military defense or homeland security applications, or, alternatively, for any civilian application, including detection of agents in buildings, post offices, ventilation ducts, carpet, clothes and electronic equipment and the like.

The samples can comprise a biological fluid (liquid) or gas taken from a human, animals or a plant, or a biological sample modified into a fluid (liquid) or gas sample. A biological fluid, or a solid biological sample, can be modified (processed) into a fluid (liquid) or a gas sample. Samples can comprises plasma, serum, blood, tears, cerebral spinal fluid (CSF), urine, saliva, semen, stool, mucus, sputum or a solution comprising isolated, cultured, disrupted or dissolved cells or tissue.

The at least one inlet port, switches, sample wells and evacuation ports can be configured and sized to handle and move samples in a nano-liter volume range, or in a microgram (μg) to nanogram (ng) volume range. The device can be operably linked to a device for automatically withdrawing one or more precise micro- or nano-liter volumes of a sample from an animal or a plant, and delivering the sample to the at least one inlet port of the device.

In alternative embodiments of the devices (products of manufacture) of the invention, the samples are modified, processed or treated, e.g., the samples can comprise a PET probe, e.g., using a PET probe comprising 2-deoxy-2-[18F]fluoro-D-glucose ([18F]FDG) (“FDG”) or equivalent in microgram (μg) to nanogram (ng) levels.

In one aspect, the at least one (or plurality of) inlet port(s), switches, sample well(s) and evacuation port(s) are configured and sized to handle and move sample(s) in a volume of about 1, 2, 3, 4, 5, 10, 20, 30, 40, 50, 100, 150, 200, 250, 300, 350, 400, 450, 500, 550, 600, 650, 700, 750, 800, 850, 900, 950 or 1000 or more nano-liters/sample or microliters per sample. The at least one (or plurality of) inlet port(s), switches, sample well(s) and evacuation port(s) can be configured and sized to handle and move samples in a volume of about at a rate of two samples per second.

The device can further comprising a pump and/or a pressure infusion tank operably linked to the device for moving the gas or fluid sample through the at least one inlet port, the switches, sample wells and/or evacuation ports. The at least one (or plurality) of the switches can be a binary (open or closed) switch, or a more complex switch capable of partial diversion to a plurality of channels.

The resin microfluidic sample device can comprise a resin, e.g., a siloxane, such as a poly(dimethyl-siloxane) (PDMS); thus, in one aspect the product of manufacture of the invention is a PDMS microfluidic sample device. The resin of the microfluidic sample device can be bonded to glass, silicon, or an equivalent substrate.

In one aspect, a multiplicity of samples of precisely metered volumes can be collected in individual wells and can be retrieved for analysis. The device can be configured to simultaneously handle at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more samples. Thee device can be configured to simultaneously and/or consecutively assay at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more samples.

In one aspect, the device is remotely controlled by a user-friendly interface operably linked to and/or integrated within the computer and the software. The device can be programmed via the user-friendly interface and/or the computer and software to take samples and/or process samples at specific time intervals. The device, and any or all components therein, can be fully or partially automated via the computer and software.

In one aspect, the at least one inlet port comprises a horseshoe shaped well with multiple channel connections, or the device further comprises a horseshoe shaped well with multiple channel connections, and the multiple channel connections are operably linked to the inlet port and/or the sample wells, and the horseshoe shaped well minimizes delay of time of diffusion of liquid or gas samples in the device. The microfluidic sample device of the invention can further comprise at least one looped channel to precisely meter the volume of flow of liquid or gas samples in the device, and the at least one looped channel is located between the input port and a sample well, and/or between a sample well and an evacuation port. In one aspect, the at least one looped channel is operably linked to a source of a purging/flushing/cleaning solution to allow cleaning or purging of the loop.

The device of the invention can further comprise a distribution node operably linked to a group of adjacent sample wells to allow the sample wells to selectively accept sample from the distribution node.

The device can further comprise at least one (or plurality of) auto-injection channel(s), e.g., wherein the at least one (or plurality of) auto-injection channel(s) are operably linked to a computer comprising enabling software.

The invention provides multiplexed systems for microfluidic sample analysis comprising the microfluidic sample device (chip) of the invention, and a device for removing sample fluids from an animal, wherein all components of the multiplexed system are operably linked to a computer comprising enabling software. The multiplexed system of the invention can comprise a device for removing sample fluids from an animal is a blood sampler, including e.g., a catheter, where the catheter can be connected to a blood sampler such that blood samples can be taken automatically without user intervention. In one aspect, the amount and timing of the blood samples is controlled by the blood sampler interfaced to a computer comprising enabling software.

The multiplexed system of the invention can be operably linked to a detection or imaging system, e.g., a CAT or a PET, such as a microPET, imaging system'. The multiplexed system can be operably linked to a computer-interface and program that controls the timing of blood collections from the animal to the microfluidic chip, and the program allows a user to specify blood sampling time intervals and number of blood samples. The multiplexed system can further comprise at least one auto-injection device, wherein the auto-injection device is separate from the sample device (chip), or is integrated into the sample device, and the auto-injection device inputs sample into the inlet port.

The multiplexed system can comprise at least one (or plurality of) auto-injection channel(s) that can be operably linked to a computer comprising enabling software.

Exemplary devices of the invention comprise those described and illustrated in FIGS. 3 to 9, and 12 to 14, and variations thereof. In one aspect, the invention provides microfabricated automatic systems to deal with reactions/reagents in a very small scale, e.g. in nano-liter range, which is an ideal characteristic for sampling blood in mouse, see the exemplary device of FIG. 3. In one aspect, the invention provides a microfluidic techniques and devices comprising use of poly(dimethyl-siloxane) (PDMS) based microfluidic systems to produce Positron Emission Tomography (PET) scans, and in one aspect use PET probes, e.g., 2-deoxy-2-[18F]fluoro-D-glucose ([18F]FDG) (“FDG”) or equivalents, in microgram (μg) to nanogram (ng) levels. This invention can be used to develop blood sampling systems on integrated microfluidic platforms to withdraw micro- and nano-liter blood samples from mice. FDG blood samples (about 250 nano-liter/sample) can be taken with a consistent volume (variation<1.5% standard deviation (s.d.)) at a rate of two samples per second.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

FIG. 1 illustrates a PET radiotracer through a mouse heart derived using manual blood sampling, as described in detail, below.

FIG. 2 graphically illustrates a typical example of the first 7 seconds of a blood time activity curves that can be used for a mouse input function in a quantitative microPET study, as described in detail, below.

FIG. 3a shows (illustrates) an exemplary device, an embodiment of an integrated microfluidic blood sampler for a mouse which can be adapted for taking samples, including fluid or gas samples, from any source, including biological sources, as described in detail, below.

FIG. 3b shows a real time snapshot of an exemplary poly(dimethyl-siloxane) (PDMS) microfluidic chip of the invention, as described in detail, below.

FIG. 4 is a schematic diagram of a ten cell embodiment of an exemplary device as illustrated in FIG. 3b, as described in detail, below.

FIG. 5 is a schematic diagram of an exemplary microfluidic sampling device of the invention, as described in detail, below.

FIG. 6 is a corresponding schematic for the embodiment illustrated in FIG. 5, as described in detail, below.

FIG. 7 is a schematic diagram of an exemplary microfluidic sampling device of the invention, as described in detail, below.

FIG. 8 illustrates a microfluidic blood sampler of the invention in a mouse quantitative microPET study, as described in detail, below.

FIG. 9 illustrates a closeup of the schematic diagram of FIG. 5, showing an exemplary microfluidic sampling device of the invention.

FIG. 10a graphically illustrates a blood curve from a mouse study using the blood sampler device design with intravenous FDG injection, as described in detail, below. FIG. 10b graphically illustrates a blood curve from a mouse study using an exemplary microfluidic sampling device of the invention, as described in detail, below.

FIG. 11a graphically illustrates image-derived blood curves using a (known) pump-driven system; FIG. 11b graphically illustrates the time (Δt) delay problem that a (known) pump-driven continuous blood drawing system has; FIG. 11c shows a result using an exemplary microfluidic chip design of the invention, as described in detail, below.

FIG. 12 shows a microPET study using a multiplexed system of the invention, including use of dynamic PET imaging using an exemplary microfluidic chip of the invention, as described in detail, below.

FIG. 13 presents images of an exemplary microfluidic chip of the invention that were obtained with microPET imaging, as described in detail, below.

FIG. 14 illustrates a closeup of the schematic diagram of FIG. 15, showing an exemplary microfluidic sampling device of the invention.

FIG. 15 illustrates an exemplary microfluidic blood sampling system of the invention comprising an auto-injection device, as described in detail, below.

FIG. 16 illustrates an overview of an exemplary microfluidic blood sampling system of the invention; FIG. 16(A) is a cartoon illustrating a blueprint of this exemplary microfluidic chip design and the connections of the chip to its operational environment; FIG. 16(B) shows an exemplary PDMS chip of the invention with the design implemented; FIG. 16(C) shows a small portion of this exemplary PDMS chip (of FIG. 16(B)); FIG. 16 (D) demonstrates the mechanism of how a valve in the control channel opens and closes a fluidic channel, as described in detail, below.

### DETAILED DESCRIPTION OF THE INVENTION

The invention provides microfabricated automatic systems comprising integrated poly(dimethyl-siloxane) (PDMS) microfluidics. The systems, including microfluidic samplers, and methods of the invention provide for withdrawing one or more precise micro- or nano-liter volumes of a sample, e.g., a sample comprising a biological fluid. The biological fluid can be taken or harvested from any subject, including humans and other mammals, such as smaller mammals where large biological fluid samples can be problematic, such as mice or other rodents. The body fluid can be, for example, urine, blood, plasma, serum, saliva, semen, stool, sputum, cerebral spinal fluid, tears, mucus, and the like.

The integrated poly(dimethyl-siloxane) (PDMS) microfluidics of the invention provide for fluidic flow and control channels that allows for the execution and automation of sequential physical, chemical, and biological processes on the same device, which in one aspect comprises digital control of the operations.

Studies described herein demonstrate the effectiveness of the microfluidic samplers of the invention in analyzing small fluid volumes, which in this exemplary embodiment comprises mouse blood samples. The studies described herein demonstrate the feasibility of deriving input functions from mice using exemplary microfluidic blood sampling devices of the invention. In the study described herein, the total blood loss (about 60 μl) and the impact on physiological changes of a mouse due to such blood loss are minimized to acceptable levels by this new invention.

Embodiments of the present invention allow the determination of input functions from small volumes of biological fluids from humans, e.g., from samples where it is difficult to get large volumes of fluid, e.g., tears, or CSF from newborns, or from forensic samples.

There is a growing interest in transgenic mice as popular animal models for studying human diseases. Embodiments of the present invention allow the determination of input functions from mice and bring microPET imaging to a new horizon of minimally invasive micro- and nano-volumetric physiological fluid sampling. The demand of a high-speed blood sampler is very high around the world, especially where technologies are moving very rapidly in producing transgenic mice.

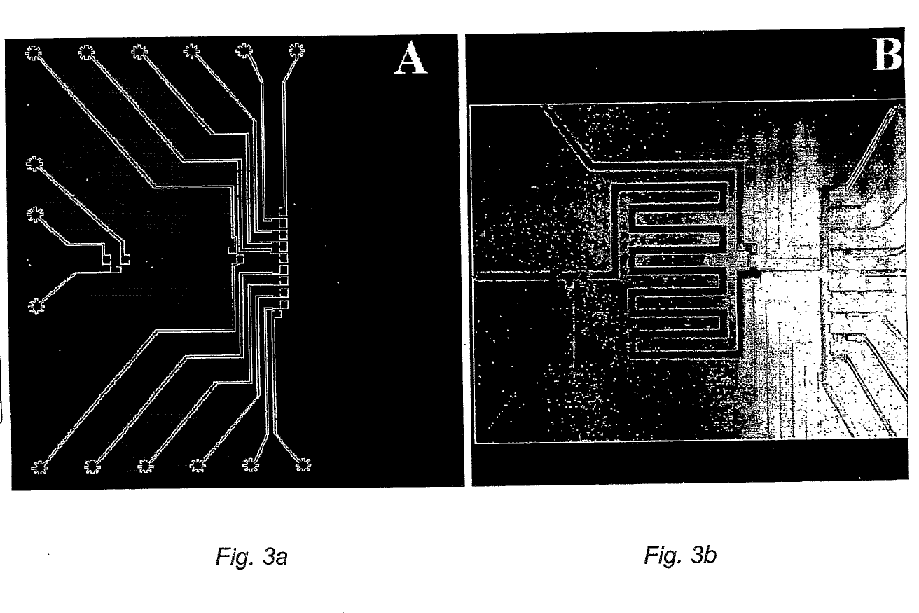

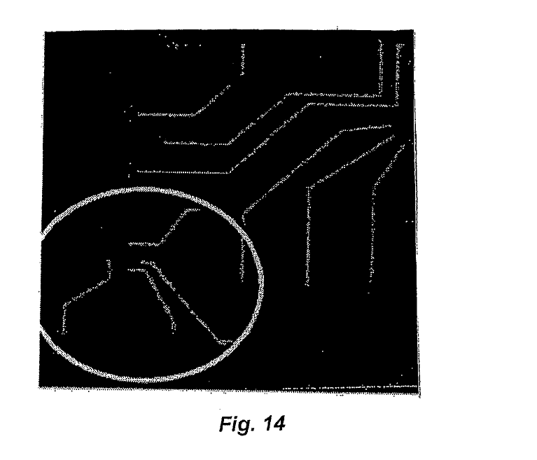

Exemplary devices comprise those described and illustrated in the Figures set forth herein, including FIGS. 3 to 9, and 12 to 14, and variations thereof.

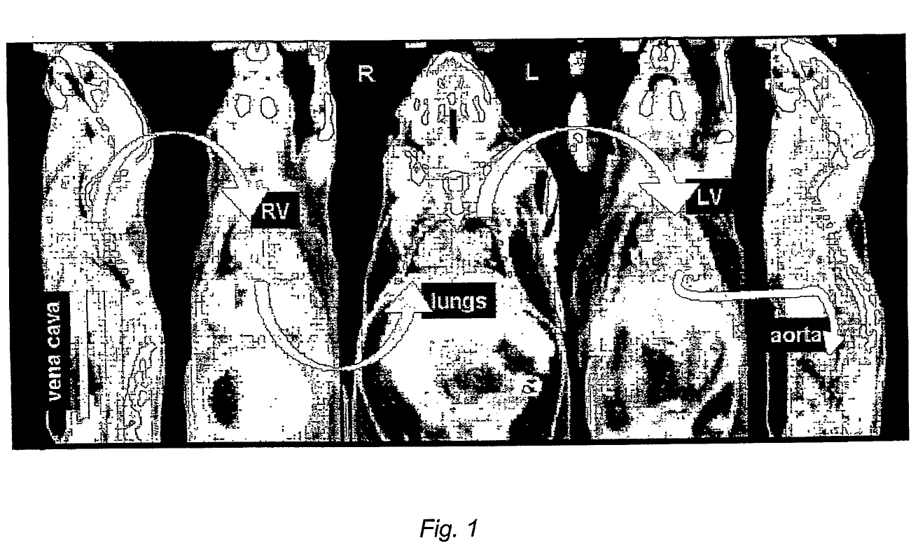

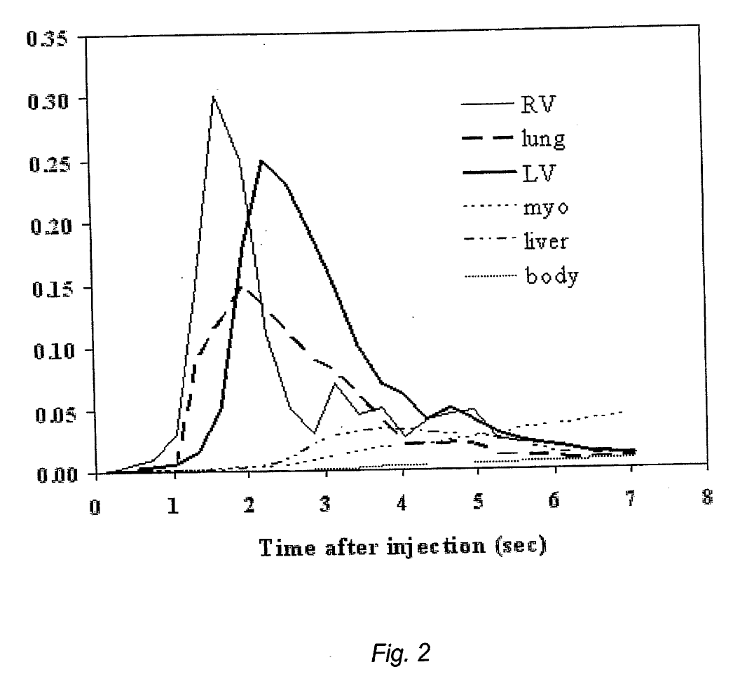

In one aspect, devices of the invention can process multiple blood samples taken within a second; this embodiment solves the problem illustrated in FIG. 1, derived using manual blood sampling, illustrating that a fast transit from vena cava (RV to aorta LV took place within 3 seconds) of a PET radiotracer through a mouse heart makes input function derivation from manual blood sampling difficult. Using this embodiment of the invention, blood time activity curves that can be used for input function in a quantitative microPET study can be processed; noting that FIG. 2 shows a typical example of the first 7 seconds of blood time activity curves that can be used for a mouse input function in a quantitative microPET study, and in order to determine the shape of each curve, multiple blood sample need to be taken within a second (as provided for by this invention).

In one aspect, the invention comprises integrated poly(dimethyl-siloxane) (PDMS) microfluidics; this technology provides for fluidic flow and control channels that allows for the execution and automation of sequential physical, chemical, and biological processes on the same device with digital control of the operations. In particular, the elasticity of PDMS materials can enable a parallel fabrication of the micro- and nano-scale functioning modules, such as valves, pumps, and columns.

Additionally, the fabrication of devices of the invention using this technology requires only relatively simple facilities. The fluidic and control networks are mapped using standard software and computer assisted detection (CAD) systems, e.g., CAD software (e.g., CAD Australia Pty. Limited.), including computer-aided design/computer-aided manufacturing (CAD/CAM) technology; and transferred onto a transparent photomasks (see, e.g., U.S. Pat. Nos. 6,982,134; 6,340,543; 6,165,649).

Photolithographic techniques can be used to make the resin-based or siloxane-based, e.g., PDMS resin-based, devices of the invention, see, e.g., U.S. Pat. Nos. 7,111,635 (fabricating a constriction region in a channel of a microfluidic device); 6,932,951; 6,752,966 (microfabrication methods and devices with microscale structural elements in an intermediate polymer layer between two planar substrates); 5,965,237; 5,534,328.

Because poly(dimethylsiloxane) (PDMS) has a hydrophobic nature, in some applications treatment of the PDMS may be desirable, e.g., the PDMS can be made hydrophilic using a simple air plasma treatment; or, for the generation of hydrophilic PDMS with long-term stability in air—a two-step extraction/oxidation process, can be used: first, PDMS is extracted in a series of solvents designed to remove unreacted oligomers from the bulk phase; second, the oligomer-free PDMS is oxidized in a simple air plasma, generating a stable layer of hydrophilic SiO₂, see, e.g., Vickers (Oct. 5, 2006) Anal, Chem., ASAP Article 10.1021/ac0609632 S0003-2700(06)00963-2. In some aspects, the device of the invention is made of mixed, or layered materials, e.g., ultraviolet light can be used to polymerize mixed monomer solutions onto the surface of a PDMS microdevice of the invention, e.g., monomers with different chemical properties can be used. In some aspects, the device of the invention can be treated by ionization of silanol groups to improve wettability. See, e.g., Hu (2003) Electrophoresis 24:3679-3688.

Any PDMS prepolymer can be used to manufacture devices of the invention, e.g., SYLGARD 184™ (Dow Corning, Midland, Mich.). PDMS polymer fabrications can be backed onto another material, e.g., a silicon nitride-coated silicon wafers (e.g., WAFERNET™, San Jose, Calif., USA).

In one aspect, photo-lithographic techniques are used to produce a reusable mold onto which a PDMS resin is poured and cured by baking. Access to the fluidic flow and control channels can be achieved by punching holes into the fabricated devices using hypodermic needles, trochars, or the like. The fabricated devices can be readily bonded to glass, silicon, or similar substrates using a variety of techniques that are well known to one of ordinary skill in the art. Large arrays of active components, such as valves and pumps, can be created by stacking and bonding multiple, individually fabricated layers. When pressurized with air or other gas mixtures or gases, a channel on a control layer that crosses a channel on the flow layer is deflected, sealing a flow channel and stopping fluid movement therein. This method of valve operation comprises binary switches (e.g., open or closed) of the microfluidics chip.

FIG. 3a shows an exemplary device, an embodiment of an integrated microfluidic blood sampler for a mouse, which in alternative aspects of the invention can be adapted for taking samples, including fluid or gas samples, from any source, including biological sources. In one exemplary embodiment of the invention, a multiplicity of blood samples (e.g., two, three, four, five, six, seven, eight, nine or ten or more) of precisely metered volumes can be collected in individual wells, and can be retrieved to undergo further analysis. The figure illustrates channels through which samples, including fluid or gas samples, are channeled from the wells. The chip can be flushed and/or purged with a medium (e.g., Heparin Lock Flush Solution, USP; e.g., HepFlush®-10. Hep-Lock® U/P; Hep-Pak® Lock Flush) between sample collections for sample purging and/or cleaning.

This exemplary device, or any device of the invention, can be remotely controlled by a user-friendly interface and can be programmed to take samples at specific time intervals.

Both saline solution and blood sample with PET probes, such as FDG-comprising blood samples, e.g., at approximately 250 nano-liter/sample, can be taken with a consistent volume (variation<1.5% s.d.) at a rate of at least one sample per second. Although the volumes are small, the PET probe (e.g., FDG) activities in blood samples are detectable and consistent (<1.2% s.d.).

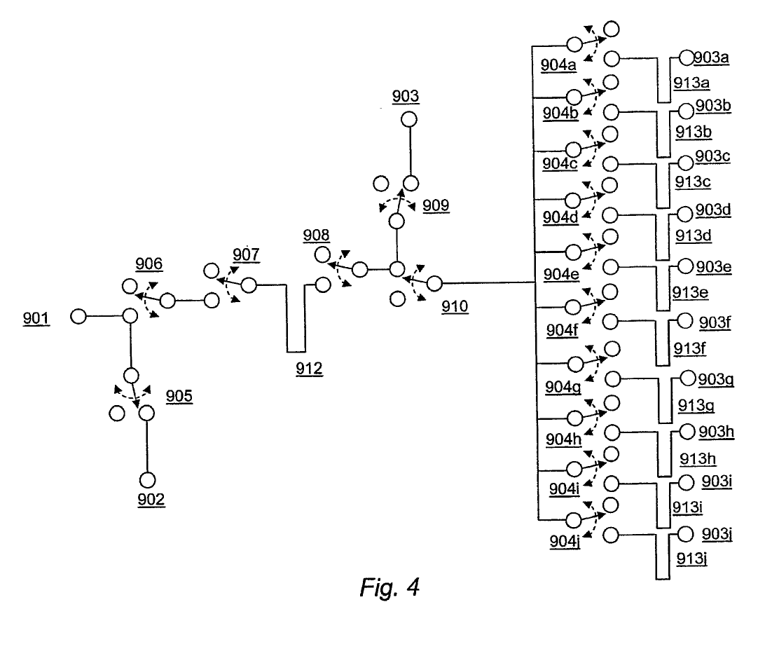

The feasibility of measuring a high precision input function for a mouse using an embodiment of this exemplary microfluidic blood sampling device has been demonstrated. FIG. 3b shows a real time snapshot of the poly(dimethyl-siloxane) (PDMS) microfluidic chip taken from an test assay. FIG. 4 is a schematic diagram of the ten cell embodiment. Fluid samples are aspirated through inlet 901 and switches 906 and 908 to volumetric metering loop 912. Switches 908, 910, 909, 903 and 905 serve to purge sample fluid from the system. Switches 904a through 904j direct a volumetrically metered sample of fluid to sample wells 913a through 913j, respectively, for PET imaging. Sample wells 913a through 913j can be evacuated through ports 903a through 903j, respectively.

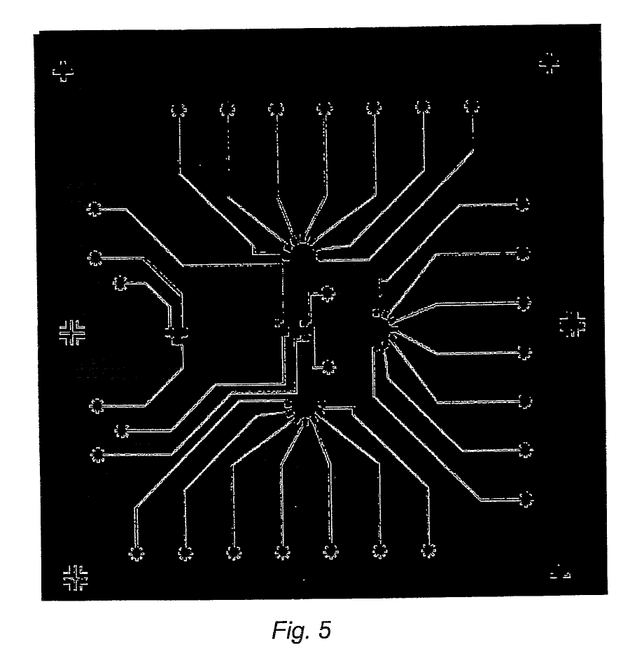

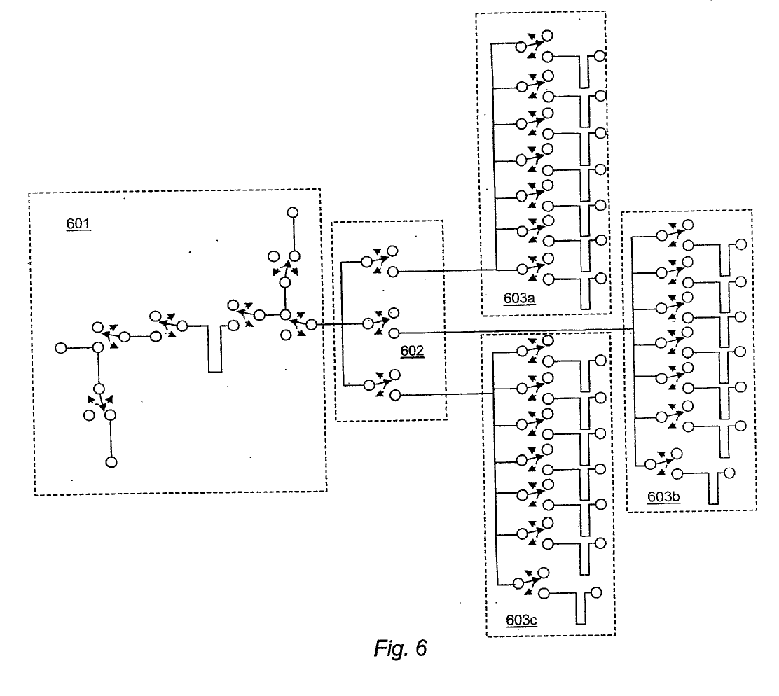

FIG. 5 shows another exemplary microfluidic sampling device of the invention; an embodiment of an exemplary design. Twenty-one blood samples can be collected on designated wells of this embodiment. FIG. 6 is a corresponding schematic for this embodiment. Block 601 circuitry is similar to the ten sample well embodiment that was discussed above. Block 602 selects one of three sample blocks 603a, 603b, and 603c, each sample block having seven selectable sample wells for MicroPET imaging.

MicroPET imaging in mice using different PET tracers (e.g. FDG, FLT, FHBG, and N-13 ammonia) can also be performed. Multiple blood samples can be taken for each mouse microPET study. An integrated software system KIS (Kinetic Imaging System) (UCLA Molecular & Medical Pharmacology Department, Regents of the University of California) can be used to assist the planning, design, and data analysis of MicroPET studies used with the methods and/or devices of this invention. The KIS system serves multiple functions—education, virtual experimentation, experimental design, and image analysis of simulated/experimental data; see, e.g., Huang (2005) Mol Imaging Biol. 7(5):330-41.

KIS is a fully integrated software system to assist the learning, planning, design, and data analysis of microPETs, e.g., microPET studies, such as mouse microPET studies. Through computer simulation and animation of tracer kinetics in all tissue organs of a whole animal (based on a realistic 3D mouse atlas), KIS allows users to learn and to evaluate conveniently a multiple of biological, chemical, and experimental factors that could affect the mouse microPET images. KIS is coded completely in Java and can be run either through the Web from a server or on a stand-alone station. With KIS, radio-tracer characteristics, administration method, dose level, imaging sequence, image reconstruction (e.g., resolution vs. noise tradeoff) can be evaluated before one actually performs a study, e.g., a mouse microPET experiment. Various kinetic data analysis procedures (including model fitting) can be examined to ensure reliable biological information can be obtained.

In one aspect, to take blood samples from an animal, e.g., a rodent (mouse or rat), or a human, a catheter can be inserted into a blood vessel of the animal, e.g., of a rodent, e.g. a femoral artery of a mouse or tail artery of a rat. Catheter implementation is a routine surgical procedure for a laboratory staff involved in a rodent study that requires drug introduction or blood sampling. Once a catheter is connected to the blood sampler, blood samples can be taken automatically without user intervention. The amount and timing of the blood samples can controlled by the blood sampler interfaced to a laptop. A user friendly program can allow a user to determine the timing and numbers of the blood samples. For further analysis of the blood samples, such as quantification of radioactivity or drug concentration in the blood, the transferring of the blood samples collected in the microfluidic chip sample wells to other containers (e.g. test tubes or Eppendorf® tubes) can controlled by a computer interface of blood sampler.

In conjunction with the quantitative microPET imaging of a test subject (for example, a mouse), an alternative method can be used to quantify the radioactivities in the blood samples. After blood collections, the microfluidic chip with blood samples can be dissembled from the unit, scanned and quantified using a microPET scan procedure.

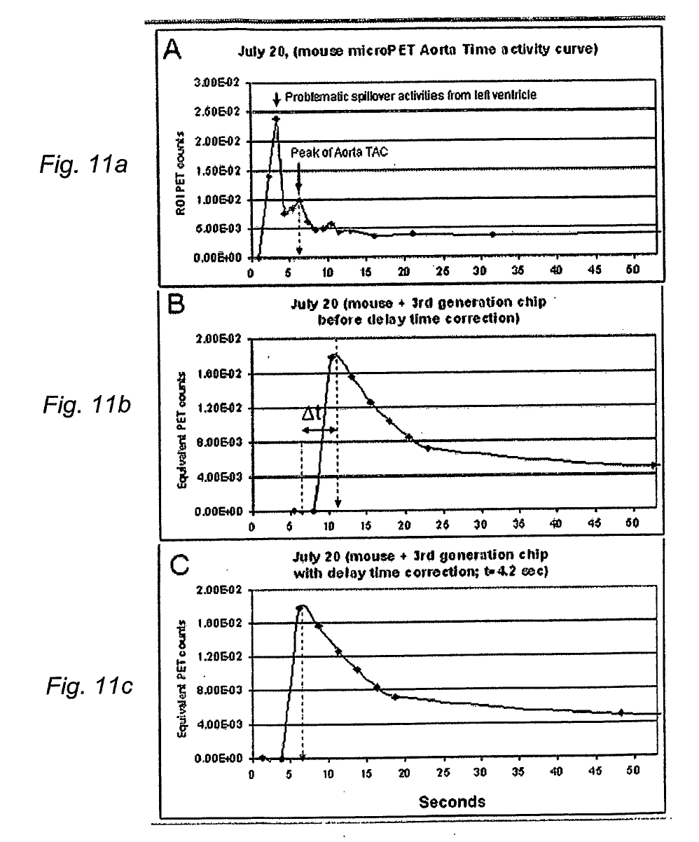

Due to difficulties and challenges with prior manual blood sampling procedures in mice, there is no precise standard in the literature to compare or validate a post-injection blood concentration curve from a blood sampler. Before this invention, most of the blood samples from rodent studies were from such manual blood drawings. We have been conducting mouse blood sampling procedures using manual drawings as controls for microPET quantitative imaging studies. We compared the blood samples analyzed using microfluidic chip devices of this invention with those from manual drawing, analyzing by a microPET-image-derived blood curve. The post-injection blood concentration curves obtained from the blood sampler are much more accurate (e.g. without dead space and time delay problems) and reproducible results compared with the prior method results. The peak and the shape of the FDG blood curves (e.g. FIG. 11c) from an embodiment of the invention have never been achieved or shown in the literature.

In one aspect, a novel feature comprises the application of microfluidic technology to in vivo biological studies that require frequent and repetitive blood sampling, for example, in a rodent (mouse or rat) study for which prior blood sampling techniques are challenging due to many reasons such as discussed above.

In one aspect, a computer-interface and program can control the timing of blood collections in the microfluidic chip. In one aspect, a user friendly program can be incorporated to allow a user to specify blood sampling time intervals and number of blood samples.

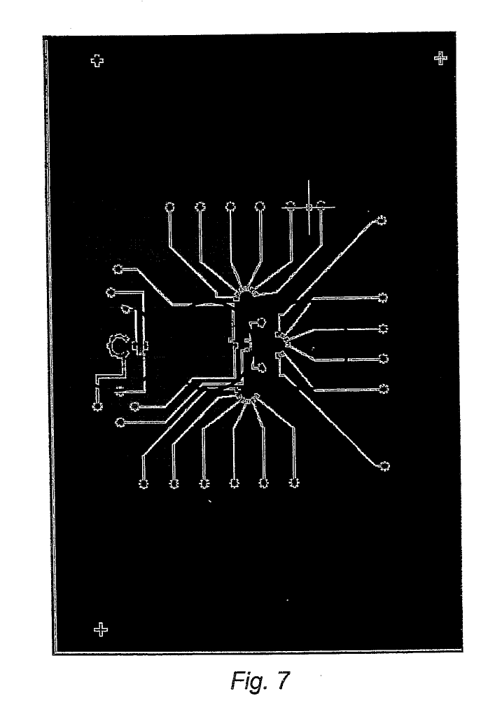

Problems of excessive time delay, dead space, and sample contamination due to diffusion, are minimized and incorporated in the exemplary microfluidic chip design, e.g., as illustrated in FIG. 7. In this illustration, the left-hand (yellow) circle designates a horseshoe shaped well with multiple channel connections to minimize the delay time and diffusion of blood samples from the withdrawal site to the chip. This site can serve two purposes, among others: (1) blood samples in a micro- to nano-liter range can be taken from the output of this site for other purposes (e.g. metabolite analysis); (2) the blood within the tube connecting the blood vessel and the chip (i.e. the dead space) can be quickly removed prior to each blood sampling by purging or flushing, as discussed above. The middle (or green) circle designates a looped channel to precisely meter the volume of each blood sample (in the micro- to nano-liter range). Connection of the loop to the purging/flushing/cleaning solution allows the cleaning or purging of the loop and avoids contamination from previous samples taken. The right hand (or magenta) circle denotes a group of adjacent sample wells to selectively accept blood from a distribution node. The distances among sample wells are maximized to minimize the partial volume effect (i.e. spillover activity) for PET imaging used to measure the blood sample radioactivities.

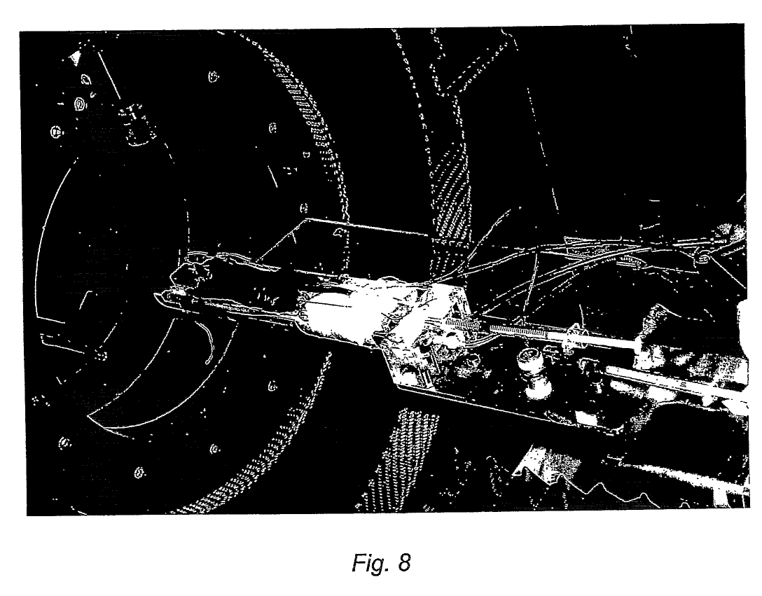

In conjunction with quantitative microPET imaging in rodent, the radioactivities of the blood samples can be quantified directly using microPET imaging. FIG. 8 illustrates a mouse quantitative microPET study using an embodiment of the microfluidic blood sampler of the invention to collect blood samples. Because of the small blood sample volume (down to a nanoliter range) collection, the blood loss due to blood sampling can be minimized and be negligible. A rodent can be studied under a stable physiological condition in vivo.

The fast blood sample collection capability (multiple snapshot samples at the first 10 seconds post-injection) enables the determination of an accurate blood concentration curve (input function) that is not achievable by manual drawing. The automation of the blood sampling procedure also minimizes necessary human intervention. In a microPET study, the automation minimized the radiation exposure to technologist/investigators, with attendant reductions in health risks.

In conjunction with microPET imaging and using the methods and devices of the invention, a true blood time activity curve (i.e. input function) can be obtained from an animal study, e.g., a rodent study, and make quantitative microPET imaging (e.g. a true functional imaging technique that provides physiological meaningful indexes) feasible.

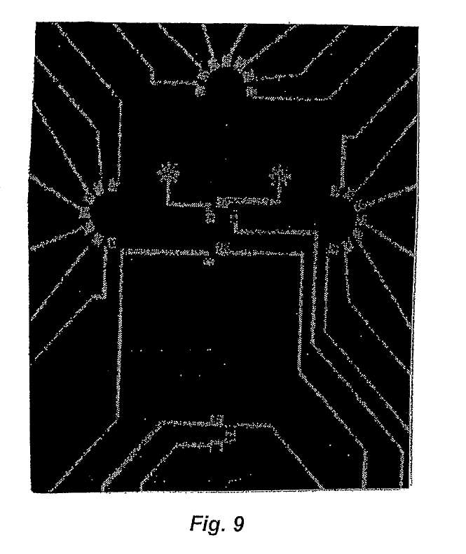

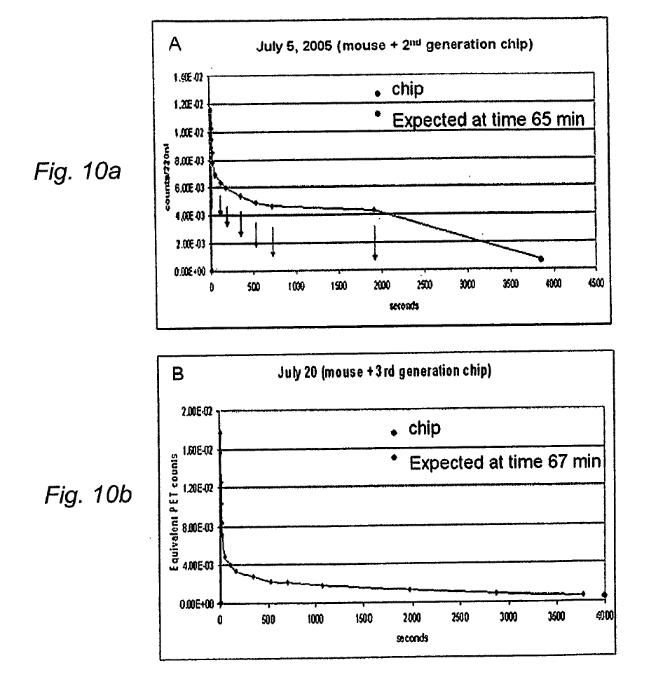

FIG. 9 is an example illustrating a previous blood sampler design by a group in University of Sherbrooke, Canada, showing a syringe pump operatively linked to a sampler (intaking medicinal air) operatively linked to a detector. FIG. 10a is a blood curve from a corresponding mouse study with intravenous FDG injection. The green arrows indicate overestimated FDG concentrations in each blood sample due to the blood remained in the catheter from a previous blood sample (the dead space problem). FIG. 10b illustrates how an exemplary design of this invention overcomes and/or eliminates the catheter dead space blood contamination.

FIG. 11a illustrates two problems that were found in image-derived blood curves: (i) spillover activities (“problematic spillover activities from left ventricle”) (red arrow); and (ii) underestimation of FDG concentration. FIG. 11b illustrates the time (Δt) delay problem that a pump-driven continuous blood drawing system has. As compared to the aorta curve (“peak of aorta TAC”) (green arrows in FIG. 11a), the curve of FIG. 11b was delayed and shifted to the right. FIG. 11c shows a result using an exemplary microfluidic chip design of the invention, indicating that it was demonstrated that use of the device of the invention resolved the time delay problem and obtained a true concentration curve.

A precise and constant blood volume (in the range of micro- to nano-liters) of each blood sample eliminates the need for blood volume normalization. For a traditional manual-drawing method, each blood example taken manually can have a different volume. Such blood samples need to be weighted using a precision scale and normalized to a fixed volume for radioactivity comparisons.

In exemplary aspects of the invention, precise digital control allows samples can be taken at rates that were not possible with prior blood sampling techniques. In addition, digital control also removes uncertainty in the time domain. The time can be accurate in the millisecond range.

In conjunction with the microPET imaging of a test subject, the radioactivities in the blood samples can be estimated by direct microPET imaging of the microfluidic chip. Therefore, prior quantification procedures that used phantom studies and scintillation well counters required by the traditional methods can be eliminated.

In one aspect, the core of a fluid sampler of the invention, e.g., a blood sampler, is the microfluidic chip, e.g., as illustrated in the Figures. Additional embodiments of the invention can use other microfluidic technologies, and these are well known to one of ordinary skill in the art, to fabricate a similar small scale of microfluidic channels, with a similar actuation system (to open and close valves between flow channels within the chip), are to be considered as part of the present invention—especially if they include the dead space elimination, and time delay of sample taking measures as described above. A supporting framework that can position and support the blood sampler but without interfering with a concurrent biological study (e.g. quantitative microPET imaging of a test subject) is also part of the present invention.

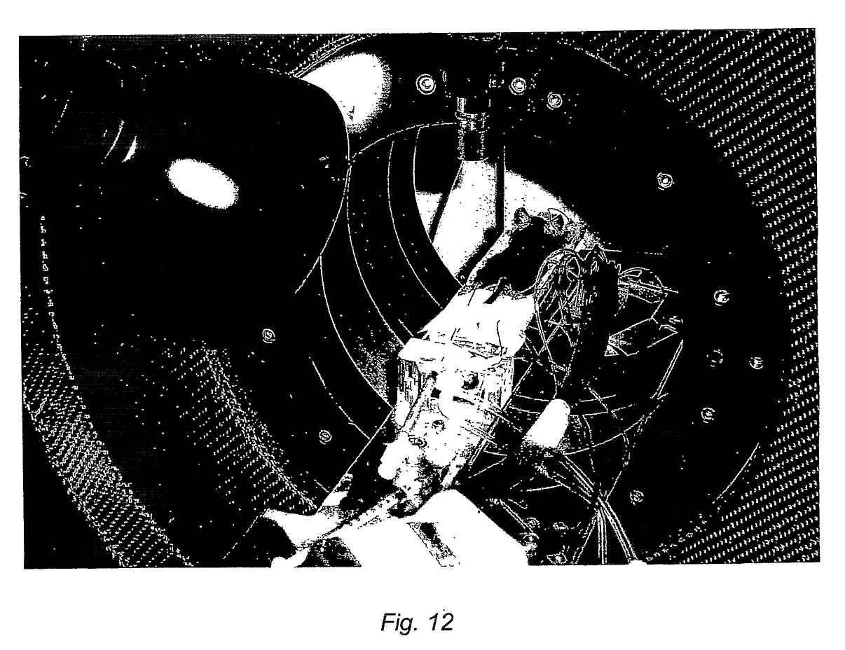

As mentioned previously in conjunction with microPET imaging above, a true blood time activity curve (i.e. input function) can be obtained from a rodent study and make quantitative microPET imaging (e.g., a true functional imaging technique that provides physiological meaningful indexes) feasible. Since the radioactivities in the blood samples can be estimated by direct microPET imaging of the microfluidic chip, calibration procedures using phantom studies and scintillation well counters required by the traditional methods can be eliminated. FIG. 12 shows a microPET study with dynamic PET imaging with a microfluidic chip according to an embodiment of the invention. Simultaneous mouse and chip imaging avoid a calibration comparison between PET scanner and well counter.

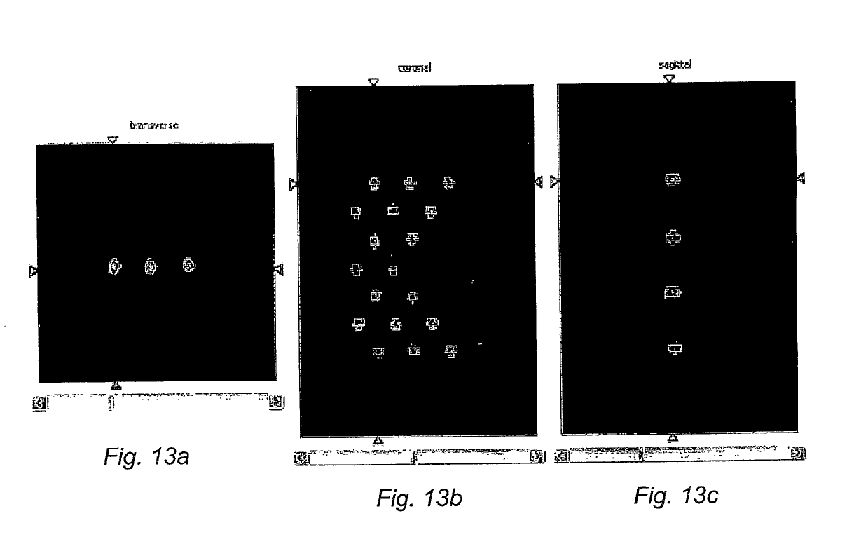

FIG. 13 presents images of a microfluidic chip according to an embodiment of the invention that were obtained with microPET imaging. The blue dots show the ¹⁸FDG samples that were collected from a ¹⁸FDG solution that has similar concentration as the dose that was injected to the mouse. The characteristics of small blood volume and fast timing precision of the blood sampler can provide an application in study of human diseases, especially for blood tests in premature babies and infants.

The invention also provides methods for using the devices of the invention. As discussed above, any sample can be analyzed by a device of the invention, including samples from biological sources. The initial sample can be solid, liquid or gas; and a sample can be prepared and/or manipulated, e.g., converted to a fluid sample, dissolved, diluted and the like, before placing into a device of the invention for analysis. The sample can be any biological sample, including those taken directly from an individual or a plant. The samples can be from microorganisms. The samples can be previously isolated or derived from an individual, e.g., from a forensic sample, a preserved sample or a histologically prepared sample.

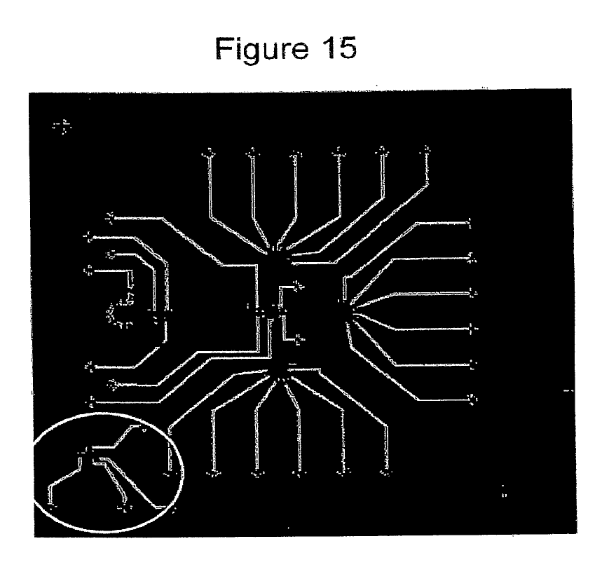

The invention also provides a device further comprising an auto-injection micro-channels, as illustrated in FIG. 15; the lower left hand (yellow) circle highlights the illustrated exemplary auto-injection component; it automates the injection of sample, including a radioactive imaging probe required by PET imaging. (Currently, all the injection methods in the PET field are either using manual injection (>95%) or a mechanical perfusion pump (<5%)).

In this embodiment, the auto-injection channels are implemented in the same chip and controlled by the same controlling system, thus, injection of sample into the device (injection with blood sampling) can be synchronized, e.g., by operative linking of the auto-injection channel(s) with the systems computer.

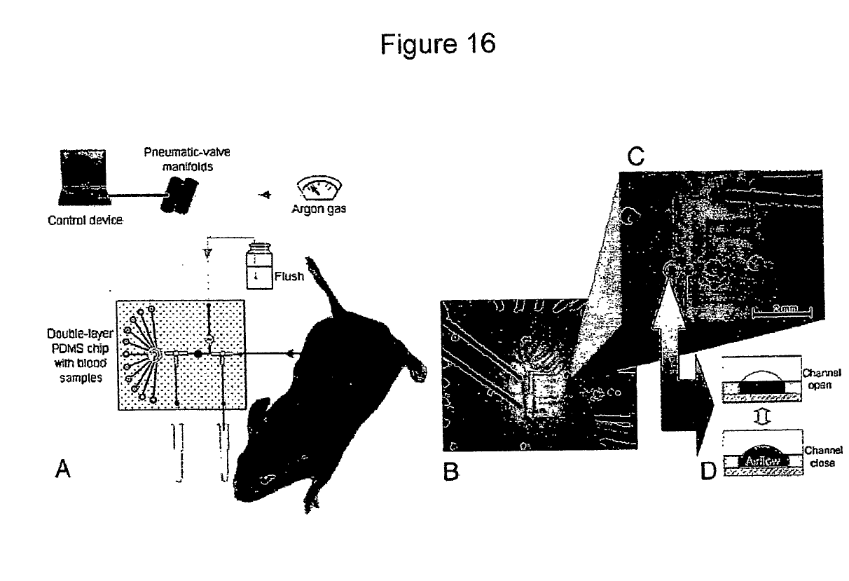

FIG. 16 illustrates an overview of an exemplary microfluidic blood sampling system of the invention. FIG. 16(A) is a cartoon illustrating a blueprint of this exemplary microfluidic chip design and the connections of the chip to its operational environment. There are two major layers in the chip. The lines (black lines) in the chip (connected to the horseshoe-shaped well) are channels in the fluidic layer. The switches in the control layer (blue) control the blood flow and flush within the fluidic channels. There is one on/off switch (circle with a minus sign), two 3-way switches (T shape) and one 18-way switch (circle with an arrow). IN this exemplary device, there are 18 blood sample channels (black lines with red wells) although only 11 channels are shown here for clarity. The illustrated image in FIG. 16(B) shows an exemplary PDMS chip of the invention with the design implemented. The control channels were filled with blue dye when the image was taken to highlight them. The black metal pins connect the control channels to the pneumatic-valve manifolds. These connections were shown in FIG. 16(A) (yellow lines). The illustrated image in FIG. 16(C) shows a small portion of this exemplary PDMS chip (of FIG. 16(B)). The 10 valves in the end of the control channels are the real parts that perform the functions of an on/off switch and a 3-way switch. The illustrated image in FIG. 16 (D) demonstrates the mechanism of how a valve in the control channel opens and closes a fluidic channel.

### Computer Systems and Computer Program Products

The devices of the invention comprise computers, and computer program products comprising a machine-readable medium including machine-executable instructions, computer systems and computer implemented methods to practice the methods and use the devices of the invention. For example, the invention provides microfluidic sample devices operably linked to a computer comprising software to control the amount of liquid or gas sample flowing through the device and/or to control movement of liquid or gas samples in the device. The computers, and computer program products comprising a machine-readable medium including machine-executable instructions also can control: the frequency and amount of sample drawn from an individual (e.g., blood samples from a mouse); the frequency and amount of sample inputted into the device of the invention; the movement of sample (liquid or gas) in the device, e.g., by controlling the rate of flow in the channels and components; the timing, amount and direction of movement of “flushing” or cleaning liquids; the timing of outputting of sample from the device; providing control access to all these modes of action to an operator, which can be in real time or automated; providing data storage; providing output and/or visualization to an operator; providing machine-readable medium including machine-executable instructions to analyze the data, and to present the analyzed data to a user.

Thus, the invention provides computers, computer systems, computer readable mediums, computer programs products and the like having recorded or stored thereon machine-executable instructions to practice the methods of the invention. The words “recorded” and “stored” can refer to a process for storing information on a computer medium. A skilled artisan can readily adopt any known methods for recording information on a computer to practice the methods and use the devices of the invention. The methods of the invention can be practiced using any program language or computer/processor and in conjunction with any known software or methodology.

Another aspect of the invention is a computer readable medium having recorded thereon machine-executable instructions to practice the methods and use the devices of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

The computer/processor used to practice the methods and use the devices of the invention can be a conventional general-purpose digital computer, e.g., a personal “workstation” computer, including conventional elements such as microprocessor and data transfer bus. The computer/processor can further include any form of memory elements, such as dynamic random access memory, flash memory or the like, or mass storage such as magnetic disc optional storage. For example, a conventional personal computer such as those based on an Intel microprocessor and running a Windows operating system can be used. Any hardware or software configuration can be used to practice the methods of the invention. For example, computers based on other well-known microprocessors and running operating system software such as UNIX, Linux, MacOS and others are contemplated. As used herein, the terms “computer,” “computer program” and “processor” are used in their broadest general contexts and incorporate all such devices.

Variations and extensions of the embodiments described are apparent to one of ordinary skill in the art. Other applications, features, and advantages of this invention will be apparent to one of ordinary skill in the art who studies this invention specification.

The details of one or more embodiments of the invention are set forth in the accompanying drawings and the description set forth herein. Other features, objects, and advantages of the invention will be apparent from the description and drawings, and from the claims.

All publications, patents, patent applications, GenBank sequences and ATCC deposits, cited herein are hereby expressly incorporated by reference for all purposes. A number of embodiments of the invention have been described. Nevertheless, it will be understood that various modifications may be made without departing from the spirit and scope of the invention. Accordingly, other embodiments are within the scope of the following claims.

1. A microfluidic sample device comprising:
(a) at least one inlet port for a fluid or a gas sample;
(b) a plurality of switches operably linked to the inlet by channels providing for fluidic flow to move the fluid or gas sample, wherein the switches can direct a volumetrically metered sample of fluid to a sample wells;
(c) a plurality of sample wells operably linked to the plurality of switches by channels providing for fluidic flow to move the fluid or gas sample;
(d) a volumetric metering loop operably linked to at least one of the switches by channels providing for fluidic flow to move the fluid or a gas sample, wherein the volumetric metering loop can purge sample fluid from the system; and,
(e) a plurality of evacuation (output) ports operably linked to the sample wells by channels providing for fluidic flow to move the fluid or gas sample.

2. The microfluidic sample device of claim 1, wherein the microfluidic sample device comprises a resin material.

3. The microfluidic sample device of claim 1, wherein the switches channel a volumetrically metered sample of fluid to sample wells.

4. The microfluidic sample device of claim 1, wherein the device is operably linked to an imaging device such that sample in the sample wells can be imaged.

5. The microfluidic sample device of claim 4, wherein the imaging device comprises a Positron Emission Tomography (PET) imaging device.

6. The microfluidic sample device of claim 1, wherein the device is operably linked to a computer comprising software to control the amount of liquid or gas sample flowing through the device and/or to control movement of liquid or gas samples in the device.

7. The microfluidic sample device of claim 1, wherein the device comprises a configuration as set forth in FIGS. 3 to 9, and FIGS. 12 to 14, or any combination thereof.

8. The microfluidic sample device of claim 1, wherein the samples comprise a biological fluid or gas.

9. The microfluidic sample device of claim 1, wherein the samples comprise a biological fluid (liquid) or gas taken from a human, an animals or a plant, or a biological sample modified into a fluid (liquid) or gas sample.

10. The microfluidic sample device of claim 1, wherein the biological fluid, or biological sample modified into a fluid (liquid) or gas sample, comprises plasma, serum, blood, tears, cerebral spinal fluid (CSF), urine, saliva, semen, stool, mucus, sputum or a solution comprising isolated, cultured, disrupted or dissolved cells or tissue.

11. The microfluidic sample device of claim 1, wherein the at least one inlet port, switches, sample wells and evacuation ports are configured and sized to handle and move samples in a nano-liter volume range, or in a microgram (μg) to nanogram (ng) volume range.

12. The microfluidic sample device of claim 1, wherein the device is operably linked to a device for automatically withdrawing one or more precise micro- or nano-liter volumes of a sample from an animal or a plant, and delivering the sample to the at least one inlet port of the device.

13. The microfluidic sample device of claim 1, wherein the samples comprise a PET probe.

14. The microfluidic sample device of claim 1, wherein the PET probe comprises 2-deoxy-2-[18F]fluoro-D-glucose ([18F]FDG) (“FDG”) or equivalent in microgram (μg) to nanogram (ng) levels.

15. The microfluidic sample device of claim 1, wherein the at least one inlet port, switches, sample wells and evacuation ports are configured and sized to handle and move samples in a volume of about 5, 10, 20, 30, 40, 50, 100, 150, 200, 250, 300, 350, 400, 450, 500, 550, 600, 650, 700, 750, 800, 850, 900, 950 or 1000 or more nano-liters/sample.

16. The microfluidic sample device of claim 1, wherein the at least one inlet port, switches, sample wells and evacuation ports are configured and sized to handle and move samples in a volume of about at a rate of two samples per second.

17. The microfluidic sample device of claim 1, further comprising a pump and/or a pressure infusion tank operably linked to the device for moving the gas or fluid sample through the at least one inlet port, the switches, sample wells and/or evacuation ports.

18. The microfluidic sample device of claim 1, wherein at least one of the switches is a binary (open or closed) switch.

19. The microfluidic sample device of claim 2, wherein the resin microfluidic sample device is a poly(dimethyl-siloxane) (PDMS) microfluidic sample device.

20. The microfluidic sample device of claim 2, wherein the resin microfluidic sample device is bonded to glass, silicon, or an equivalent substrate.

21. The microfluidic sample device of claim 1, wherein a multiplicity of samples of precisely metered volumes can be collected in individual wells and can be retrieved for analysis.

22. The microfluidic sample device of claim 1, wherein the device is configured to simultaneously handle at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more samples.

23. The microfluidic sample device of claim 1, wherein the device is configured to simultaneously assay at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more samples.

24. The microfluidic sample device of claim 6, wherein the device is remotely controlled by a user-friendly interface operably linked to and/or integrated within the computer and the software.

25. The microfluidic sample device of claim 24, wherein the device is programmed via the user-friendly interface and/or the computer and software to take samples and/or process samples at specific time intervals.

26. The microfluidic sample device of claim 1, wherein the at least one inlet port comprises a horseshoe shaped well with multiple channel connections, or the device further comprises a horseshoe shaped well with multiple channel connections, and the multiple channel connections are operably linked to the inlet port and/or the sample wells, and the horseshoe shaped well minimizes delay of time of diffusion of liquid or gas samples in the device.

27. The microfluidic sample device of claim 1, wherein the device further comprises at least one looped channel to precisely meter the volume of flow of liquid or gas samples in the device, and the at least one looped channel is located between the input port and a sample well, and/or between a sample well and an evacuation port.

28. The microfluidic sample device of claim 27, wherein the at least one looped channel is operably linked to a source of a purging/flushing/cleaning solution to allow cleaning or purging of the loop.

29. The microfluidic sample device of claim 1, wherein the device further comprises a distribution node operably linked to a group of adjacent sample wells to allow the sample wells to selectively accept sample from the distribution node.

30. The microfluidic sample device of claim 1, wherein the device further comprises at least one auto-injection channel.

31. The microfluidic sample device of claim 1, wherein the at least one auto-injection channel are operably linked to a computer comprising enabling software.

32. A multiplexed system for microfluidic sample analysis comprising the microfluidic sample device (chip) of claim 1, and a device for removing sample fluids from an animal, wherein all components of the multiplexed system are operably linked to a computer comprising enabling software.

33. The multiplexed system of claim 32, wherein the device for removing sample fluids from an animal is a blood sampler and a catheter, and the catheter is connected to a blood sampler such that blood samples can be taken automatically without user intervention.

34. The multiplexed system of claim 33, wherein the amount and timing of the blood samples is controlled by the blood sampler interfaced to a computer comprising enabling software.

35. The multiplexed system of claim 32, wherein the multiplexed system is operably linked to a microPET imaging system.

36. The multiplexed system of claim 33, wherein the multiplexed system is operably linked to a computer-interface and program that controls the timing of blood collections from the animal to the microfluidic chip, and the program allows a user to specify blood sampling time intervals and number of blood samples.

37. The multiplexed system of claim 33, wherein further comprising at least one auto-injection device, wherein the auto-injection device is separate from the sample device (chip), or is integrated into the sample device, and the auto-injection device inputs sample into the inlet port.

38. The multiplexed system of claim 37, wherein the at least one auto-injection channel is operably linked to a computer comprising enabling software.
